(19) United States
(12) Patent Application Publication
(10) Pub. No.: US 2010/0183641 A1
(43) Pub. Date: Jul. 22, 2010

(54) SPERM LIGANDS AND METHODS OF USE

(75) Inventors: Kenneth L. White, North Logan, UT (US); Bart C. Weimer, Davis, CA (US); Barry Joseph Pate, Logan, UT (US); Dong Chen, North Logan, UT (US)

Correspondence Address: UTAH STATE UNIVERSITY;TECHNOLOGY COMMERCIALIZATION OFFICE, 570 RESEARCH PARK WAY, SUITE 101, NORTH LOGAN, UT, 84341, US

(73) Assignee: Utah State University, North Logan, UT (US)

(21) Appl. No.: 12/726,147

(22) Filed: Mar. 17, 2010

Related U.S. Application Data
(63) Continuation of application No. 12/002,813, filed on Dec. 19, 2007.
(60) Provisional application No. 60/870,950, filed on Dec. 20, 2006.

(51) Int. Cl. A61K 39/00; A61P 15/18

(52) U.S. Cl. 424/184.1

(57) ABSTRACT

Identified herein are sperm ligand proteins located in the membrane of sperm, which proteins interact with the membrane of oocytes. Methods of using these proteins, or fragments or derivatives or analogs thereof, are also described. These include methods of increasing (or reducing) successful fertilization, for instance through improved sperm-oocyte binding, fusion or activation (or the blocking thereof); methods of preventing fertilization of an oocyte, for instance by inducing an immune response to at least one sperm ligand that promotes sperm-oocyte binding, sperm-oocyte fusion or oocyte activation; and methods for enhancing assisted reproductive technologies, for instance through stimulation of activation with nuclear transfer, stimulation of inactive or weak sperm, and so forth.

### RELATED APPLICATIONS

This application is a continuation of U.S. patent application Ser. No. 12/002,813, filed on Feb. 7, 2008, entitled “Sperm Ligands and Methods of Use,” which claims the benefit of U.S. Provisional Application No. 60/870,950, filed Dec. 20, 2006, entitled, “Sperm Ligands and Methods of Use.” All figures of U.S. patent application Ser. No. 12/002,813, filed on Feb. 7, 2008, entitled “Sperm Ligands and Methods of Use,” and of U.S. Provisional Application No. 60/870,950, filed Dec. 20, 2006, entitled, “Sperm Ligands and Methods of Use” are hereby incorporated by reference herein.

### STATEMENT OF GOVERNMENT INTEREST

This invention was made with United States government support pursuant to grant 2002-35203-12669, from the USDA Cooperative State Research, Education, and Extension Service; the United States government has certain rights in the invention.

### FIELD OF THE DISCLOSURE

This disclosure relates to sperm-specific proteins that interact with oocyte plasma membrane proteins. It further relates to methods of their use, for instance in contraceptive systems (such as contraceptive vaccines), to improve oocyte fertilization, and to enhance sperm binding, sperm-oocyte fusion, and/or oocyte activation.

### BACKGROUND

Calcium is a divalent cation that commonly functions as a second messenger, relaying signals downstream so that a cell can respond to various stimuli. The cell strictly maintains a very low intracellular level of calcium and there are mechanisms in place that maintain this low level, including ATP driven ion channels and ion exchange channels. In this way, calcium is either sequestered within the endoplasmic reticulum or in the extracellular space. Because of the low intracellular calcium level there is a strong gradient which, when an appropriate signal is received, allows a very rapid influx of calcium down the gradient. There are several enzymes within the cell that respond to this rapid increase in calcium, including calmodulin and calpain. These enzymes can, in turn, activate other enzymes thus propagating the signal cascade.

At fertilization, the sperm triggers a series of intracellular calcium oscillations that are pivotal to oocyte activation and development in every species that has been studied (Berridge and Galione FASEB J. 2:3074-3082, 1988; Kline and Kline Dev. Biol. 149:80-89, 1992). The biological significance of the changes in Ca²⁺ᵢ concentration as it relates to oocyte activation is not fully understood, however, calcium ions are known to be involved in cortical granule release which leads to a block to polyspermy and in the control of cell cycle progression (Kline and Kline 1992).

One hypothesis to explain how sperm initiate Ca²⁺ᵢ oscillations in mammalian oocytes is that spermatozoa interact with a receptor located in the plasma membrane of the oocyte. This receptor is postulated to be coupled to a trimeric GTP-binding protein (G-protein) or to have tyrosine kinase activity and to be able to activate phospholipase C which, in turn, stimulates the production of diacylglycerol and 1,4,5 inositol trisphosphate (IP3), a common Ca²⁺ releasing compound, from phosphatidyl inositol (4,5)-bisphosphate.

Evidence in support of this receptor-mediated activation hypothesis points to the involvement of integrins. Integrin molecules are cell surface adhesion receptors which form a family of transmembrane glycoproteins with heterodimeric structure (Hynes Cell 69:11-25, 1992). Many integrins recognize the RGD amino acid sequence, which appears in extracellular matrix (ECM) proteins and cell surface molecules (Ruoslahti and Pierschbacher Science 238:491-497, 1987). Integrins facilitate attachment of the cell to the ECM, facilitate cell migration, mediate cell-cell adhesion, link the ECM with the cellular cytoskeleton, and act as two-way signaling molecules (Sjaastad and Nelson Bioessays 19:47-55, 1997). Initiation of adhesion activates ‘outside in’ signaling mechanisms, which can feedback ‘inside out’ signaling to regulate integrin function, cytoskeletal assembly, cell behavior, and protein synthesis (Hynes 1992). Integrins bind their ligands relatively loosely compared to other receptors, but are present in much higher concentrations on the surface of cells. Because of this loose binding they cluster together at the site of attachment in order to bind ligands sufficiently tightly. In addition, they are known to associate with other cell surface proteins such as members of the tetraspannin family. CD9, a tetraspannin signaling molecule known to associate with β1 integrins (Chen et al., Proc. Natl. Acad. Sci. USA 96:11830-11835, 1999), has been shown to be involved in the process of sperm-oocyte fusion. Oocytes from female CD9−/− mice were unable to fuse with sperm, and hence were infertile (Miyado et al. Science 287:321-324. 2000). The oocyte receptor, or group of receptors, is apparently quite complex and is yet to be completely understood.

Integrins have also been shown to be involved in the process of fertilization (Almeida et al. Cell 81:1095-1104, 1995; Bronson et al. Mol. Reprod. Dev. 52:319-327, 1999; Bronson and Fusi Biol. Reprod. 43:1019-1025, 1990). In 1990, Bronson and Fusi showed that addition of RGD-containing peptides in a heterologous system (human sperm and zona-free hamster oocytes) or a homologous system (hamster sperm and zona-free hamster oocytes) resulted in the complete inhibition of fertilization. In 1995, Almeida et al. characterized integrins present on the plasma membrane of unfertilized murine oocytes and showed, with a combination of antibody inhibition, peptide inhibition, and somatic cell transfection experiments, that the integrin α6β1 serves as a sperm receptor. A number of integrins and their ligands have been described on human oocytes and sperm (Klentzeris et al. Hum. Reprod. 10:728-733, 1995). Integrin subunits have also been shown to be present on mature bovine oocytes.

When integrins bind to form cell-matrix or cell-cell interactions, they cluster together. As the integrins cluster, other enzymes and proteins accumulate on the cytoplasmic face of the plasma membrane to initiate a signal. The recruitment of a cytoplasmic tyrosine kinase (CTK) called focal adhesion kinase (also known as protein tyrosine kinase 2, hereafter referred to as FAK) is characteristic of many integrin signaling pathways. Binding of integrins to intracellular elements like talin and paxillin induces the recruitment and clustering of FAK enzymes. FAK and paxillin are important components of integrin-regulated signaling. Evidence suggests that these two proteins have a role in communication across cell-matrix and cell-cell junctions. FAK is known to be involved in the regulation of N-cadherin-based cell-cell adhesion (Schaller J. Cell Biol. 166:157-159, 2004; Yano et al. J Cell Biol. 166:283-295, 2004). FAK molecules cross-phosphorylate each other on certain tyrosine residues that act as a site of attachment for various CTKs from the SRC family. SRC family kinases phosphorylate other tyrosines on FAK as well as other proteins that have been recruited to the focal adhesion, thereby activating them. FAK is considered to be a regulator of focal adhesions. Through these focal adhesions many intracellular signaling pathways are initiated (Parsons et al. Oncogene 19:5606-5613, 2000). We have previously demonstrated both the presence of FAK in mature bovine oocytes and the functional role of FAK in the process of bovine oocyte activation.

If integrins do mediate sperm-oocyte interactions, then a variety of CTKs, including FAK and the SRC family are implicated for a possible role in oocyte activation. Genistein is a commonly used inhibitor of tyrosine kinases that has been shown to inhibit EGFR, v-Src, c-Src, v-Abl, PKA, and PKC. Our data demonstrates the ability of genistein to inhibit both Ca²⁺ᵢ and development following fertilization. Tyrosine kinase involvement in oocyte activation pathways has also been detected in mouse oocytes (Mori et al. Biochem. Biophys. Res. Commun. 182:527-533, 1992), pig oocytes (Kim et al. Biol. Reprod. 61:900-905, 1999), and Xenopus eggs (Abassi and Foltz Dev. Biol. 164:430-443, 1994; Moore and Kinsey Dev. Biol. 168:1-10, 1995). Although there is a clear indication that one or more tyrosine kinases are involved, it is yet unclear which specific kinase it is, and their complete role in mammalian fertilization remains under investigation.

The largest family of cell-surface receptors in eukaryotes is the G-protein-linked receptor family. When extracellular signaling molecules bind receptors, the receptors undergo a conformational change that activates G-proteins. G-proteins are trimers composed of α, β, and γ subunits. There are several known isoforms of alpha subunits, which are used to classify the various G-protein signaling trimers. Activation of a G-protein occurs when an activated receptor induces the a subunit to exchange a bound GDP molecule for a GTP molecule. Upon binding GTP, the trimer dissociates into an a subunit and a βγ subunit. Each type of α subunit and each βγ subunit can act as a signaling molecule, targeting specific enzymes. Gs α can activate Ca²⁺ channels, while Go βγ can inactivate Ca²⁺ channels. Several subunits can also activate phospholipase isoforms.

In 1994 it was reported that injection of guanosine 5′-0-(2-thiodiphosphate) (GDPβ2), a G-protein antagonist, into fertilized rabbit oocytes resulted in inhibition of intracellular Ca²⁺ oscillations. GDPβ2 is a non-hydrolizable GDP analog that competitively inhibits G-protein activation by GTP. It has also been hypothesized that G-proteins were involved in the production of IP3 (Fissore and Robl Dev. Biol. 166:634-642, 1994). Acetylcholine, known to interact with plasma membrane-coupled G-protein receptors, and injection of GTPγ(S), an activator of G-proteins, elicits Ca²⁺ᵢ oscillations (Williams et al. Dev. Biol. 151:288-296, 1992). A study by Kim et al. (J. Physiol. 513:749-760, 1998) showed that an exogenously added rat Ml muscarinic receptor mediated porcine oocyte activation by a G-protein coupled signal transduction pathway leads to oocyte activation. More recently Zeng et al. (Curr. Biol. 13:872-876, 2003) reported that the Gβγ subunit is responsible for the modulation of IP3 binding to IP3 receptors (IP3R) and that it stabilizes IP3Rs in a channel conformation that is similar to what occurs after IP3 binding. Zeng et al. suggested Gβγ as an alternative to IP3 in activating IP3R. It also appears that G-proteins are functional in bovine oocyte development.

One of the mammalian sperm proteins thought to be involved in adhesion and fusion of gametes is fertilin. Fertilin is a heterodimeric membrane protein composed of an α and a β subunit (Blobel et al. Nature 356:248-252, 1992). The fertilin ligand has been linked to sperm-oocyte binding and fusion. Sperm from mice lacking fertilin β are deficient in their ability to adhere to and fuse with oocytes (Cho et al. Science 281:1857-1859, 1998). Fertilin β on murine sperm is also known to bind the α6β1 integrin, and requires CD9 as a co-receptor (Chen et al., Proc. Natl. Acad. Sci. USA 96:11830-11835, 1999).

Both fertilin αand β, along with snake venom disintegrins, are members of a growing family of proteins known as ADAMs (Wolfsberg et al. J. Cell Biol. 131:275-278, 1995; Wolfsberg et al. Dev. Biol. 169:378-383, 1995). To date there are 15 ADAM family members described and sequenced at the cDNA level in the guinea pig, monkey, mouse, rabbit, rat, and human (Wolfsberg and White Dev. Biol. 180:389-401, 1996). It should also be noted that fertilin α and β, formerly known as PH-30α and PH-30β, are now referred to as ADAMs 1 and 2 (Huang Cell. Mol. Life Sci. 54:527-540, 1998; Wolfsberg and White 1996). All members of this family contain five functional domains: a proteolytic domain, an adhesion domain (disintegrin domain), a fusion domain, an EGF-like domain, and a signaling domain (Wolfsberg and White 1996).

The specific identity of a disintegrin, ADAM, or other RGD containing protein on the sperm inner acrosomal membrane is still to be determined. Identification of sperm ligands and intracellular signaling molecules can be used to increase the efficiency of in vitro embryo production (for instance by nuclear transfer and other assistive technologies), increase efficiency of intracytoplasmic sperm injection (ICSI), or help in the reduction of species (or populations) in which overpopulation is a concern.

### SUMMARY

Described herein is the identification of sperm ligand proteins located in the membrane of sperm, which proteins interact with the membrane of oocytes. Methods of using these proteins, or fragments or derivatives or analogs thereof, are also described. These include methods of increasing (or reducing) successful fertilization, for instance through improved sperm-oocyte binding, fusion or activation (or the blocking thereof); methods of preventing fertilization of an oocyte, for instance by inducing an immune response to at least one sperm ligand that promotes sperm-oocyte binding, sperm-oocyte fusion or oocyte activation; and methods for enhancing assisted reproductive technologies, for instance through stimulation of activation with nuclear transfer, stimulation of inactive or weak sperm, and so forth.

In one embodiment there is provided a method of increasing oocyte fertilization, which comprises treating an oocyte with a purified sperm protein, or fragment thereof, that interacts with the oocyte plasma membrane and promotes specific binding, sperm-oocyte fusion, or oocyte activation. In various examples, the purified sperm protein comprises an integrin-binding sequence.

It is specifically contemplated that the oocyte in certain uses of the described methods is fertilized in vitro (for instance, by intracytoplasmic sperm injection) and/or the oocyte is a recipient for nuclear transfer.

In certain examples of this method, the purified sperm protein induces oocyte activation, and/or promotes sperm-oocyte fusion, and/or promotes sperm binding to the oocyte. By way of example, the purified sperm protein is a bacterial outer membrane protein-like protein in some instances.

Also provided are methods to prevent fertilization of an oocyte, which comprises inducing in a subject an immune response to at least one sperm protein that interacts with the oocyte plasma membrane and induces specific binding, sperm-oocyte fusion, or oocyte activation, such that fertilization of the oocyte is blocked. By way of example, the sperm protein in certain instances contains an integrin binding sequence. The sperm protein in certain embodiments induces specific binding of sperm to the oocyte, and/or induces sperm-oocyte fusion and/or induces oocyte activation.

In example embodiments of these methods, induction of the immune response comprises administration of at least one purified polypeptide, comprising a sperm protein that interacts with the oocyte plasma membrane, in a pharmaceutically acceptable carrier, such that an immune response sufficient to prevent fertilization is generated.

Yet other described methods are methods to prevent fertilization of an oocyte, which methods involve treating an oocyte with a purified sperm protein, or fragment thereof, that interacts with the oocyte plasma membrane and inhibits or blocks specific binding, sperm-oocyte fusion, or oocyte activation, such that fertilization of the oocyte is blocked.

By way of example, in any of the described methods, the purified sperm protein may be selected from the proteins listed in Table 1.

The foregoing and other features and advantages will become more apparent from the following detailed description, which proceeds with reference to the accompanying figure(s).

### BRIEF DESCRIPTION OF THE DRAWINGS

FIG. 1 is a series of two-dimensional gel images from a crosslinking experiment, showing binding of sperm proteins to oocyte plasma membrane. FIG. 1A is the 2-D gel image of the sperm proteins that were not bound to oocyte plasma membranes proteins. FIG. 1B is the 2-D gel image of the sperm proteins after binding to oocyte membrane proteins. FIG. 1C is the 2-D gel image of the sperm proteins after binding to oocyte membrane proteins with the addition of a cross-linking agent. FIG. 1D is the overlaid image of FIG. lB and FIG. 1C. In FIG. 1D, many protein spots from FIG. 1B and FIG. 1C overlay each other and have not changed position due to the addition of the cross-linking agent. Several spots in FIG. 1D identify proteins that have shifted due to the cross-linking agent. The observed protein shifts in FIG. lB or FIG. 1C are protein targets that may be involved in the sperm-oocyte interaction.

### DETAILED DESCRIPTION

### I. Abbreviations

ACE: angiotensin-converting enzyme

ADAM: “A Disintegrin And Metalloprotease”

BOMP: bacterial outer membrane protein

BSA: bovine serum albumin

CTK: cytoplasmic tyrosine kinase

ECM: extracellular matrix

FAK: focal adhesion kinase

HSP70: heat shock protein 70

ICSI: intracytoplasmic sperm injection IP3: 1,4,5 inositol trisphosphate (also, triphosphoinositol)

IPG: immobilized pH gradient

IVF: in vitro fertilization

LAP: leucine aminopeptidase

### II. Terms

Explanations of terms and methods are provided herein to better describe the present disclosure and to guide those of ordinary skill in the art in the practice of the present disclosure. The singular forms “a,” “an,” and “the” refer to one or more than one, unless the context clearly dictates otherwise. For example, the term “including a nucleic acid” encompasses single or plural nucleic acids, and is considered equivalent to the phrase “including at least one nucleic acid.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. For example, the phrase “mutations or polymorphisms” or “one or more mutations or polymorphisms” means a mutation, a polymorphism, or combinations thereof, wherein “a” can refer to more than one.

Although methods and materials similar or equivalent to those described herein can be used in the practice or testing of the present disclosure, suitable methods and materials are described. The materials, methods, and examples are illustrative only and not intended to be limiting. All publications, patent applications, patents, and other references mentioned herein are incorporated by reference in their entirety.

Unless otherwise noted, technical terms are used according to conventional usage. Definitions of common terms in molecular biology may be found in various technical publications, including for instance Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Bacterial Outer Membrane Protein (BOMP): A family of proteins that reside in the outer membrane of gram-negative bacteria. BOMPs have a variety of functions, including general porins, components of protein export systems, proteins involved in biogenesis of the flagella and pili, and enzymes (Koebnik et al. Mol Microbiol. 37:239-253, 2000). BOMPs appear to all have a β-barrel structure.

Crosslinking agent: A chemical that promotes the formation of chemical links between molecules to form a three-dimensional network of connected molecules. Crosslinking agents suitable for generating connections between proteins are well known to those of skill in the art of protein-protein interactions. See, e.g. Pierce Chemicals, Crosslinking Reagents: Technical Handbook, for examples and general discussions.

Crosslinking reagents include, but are not limited to, heterobifunctional, homobifunctional and trifunctional reagents, which can be used to introduce, produce or utilize reactive groups, such as thiols, amines, hydroxyls and carboxyls, on one or more molecules to form a chemical linkage between two (or more) molecules. Crosslinking agents can cause the formation of covalent bonds between proteins as they interact, allowing for the analysis of protein:protein complexes.

Fertilized/Fertilization: The union of two gametes (in animals, a sperm and an oocyte) such that a new organism (zygote) is produced. Fertilization consists of the binding of a sperm to an oocyte, the fusion of the sperm and oocyte, re-establishment of a diploid chromosome composition, and activation of the oocyte to begin the developmental program.

Integrin binding sequence: A short peptide motif that binds to (or is bound by) integrins. Most integrins bind to an amino acid sequence element that contains an aspartic acid residue. The most common integrin binding sequence is the RGD motif (arginine-glycine-aspartic acid). Other integrin binding sequences include, but are not limited to, ECD (glutamic acid-cysteine-aspartic acid), LDV (leucine-aspartic acid-valine), KGD (lysine-glycine-aspartic acid), RTD (arginine-threonine-aspartic acid), and KQAGD (lysine-glutamine-alanine-glycine-aspartic acid).

Intracytoplasmic sperm injection (ICSI): An in vitro fertilization procedure in which a sperm is injected directly into an oocyte. ICSI is frequently used to improve the pregnancy rate from IVF for oligozoospermic individuals.

In vitro fertilization (IVF): A technique in which oocytes are fertilized in a culture dish. Oocytes and sperm are incubated together in cell culture medium. Following fertilization, the resulting embryo is grown in culture, usually to the blastocyst stage, and may then be implanted in a host female for further development.

Oocyte activation: Stimulating re-initiation of the cell cycle leading to cell division in an oocyte by fertilization or artificial means. Artificial means of oocyte activation include electrical pulse, treatment with ethanol, or by treatment with a calcium ionophore, followed by addition of a protein synthesis inhibitor.

Pharmaceutically acceptable carrier: The art recognizes standard pharmaceutical carriers, including, but not limited to, water, buffered saline, oil/water emulsions, or water/oil emulsions. The carrier may contain additives such as substances that enhance isotonicity and/or chemical stability. The additive materials may include buffers such as phosphate, citrate, succinate, acetic acid, and other organic acids or their salts; antioxidants such as ascorbic acid; low molecular weight (for instance, less than about twelve residues) polypeptides, proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, trehalose, glucose, mannose, or dextrins; chelating agents such as EDTA; sugar alcohols such as mannitol or sorbitol; counter-ions such as sodium; and/or nonionic surfactants such as polysorbates, poloxamers, or PEG.

Purified: The term “purified” does not require absolute purity; rather, it is intended as a relative term. Thus, for example, a purified protein preparation is one in which the protein referred to is more pure than the protein in its natural environment within a cell or within a production reaction chamber (as appropriate).

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Homologs or orthologs of a protein, and the corresponding cDNA or gene sequence, will possess a relatively high degree of sequence identity when aligned using standard methods. This homology will be more significant when the orthologous proteins or genes or cDNAs are derived from species that are more closely related (e.g., human and chimpanzee sequences), compared to species more distantly related (e.g., human and C. elegans sequences).

Methods of alignment of sequences for comparison are well known in the art.

Various programs and alignment algorithms are described in: Smith & Waterman Adv. Appl. Math. 2: 482, 1981; Needleman & Wunsch J. Mol. Biol. 48: 443, 1970; Pearson & Lipman Proc. Natl. Acad. Sci. USA 85: 2444, 1988; Higgins & Sharp Gene, 73: 237-244, 1988; Higgins & Sharp CABIOS 5: 151-153, 1989; Corpet et al. Nuc. Acids Res. 16, 10881-90, 1988; Huang et al. Computer Appls. in the Biosciences 8, 155-65, 1992; and Pearson et al. Meth. Mol. Bio. 24, 307-31, 1994. Altschul et al. (J. Mol. Biol. 215:403-410, 1990), presents a detailed consideration of sequence alignment methods and homology calculations.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet at ncbi.nlm.nih gov/BLAST/. A description of how to determine sequence identity using this program is available on the internet at ncbi.nlm.nih gov/BLAST/blast_help.html.

Homologous nucleic acid or protein sequences are typically characterized by possession of at least 60%, 70%, 75%, 80%, 85%, 90%, 92%, 95% or at least 98% sequence identity counted over the full length alignment with a sequence using the NCBI Blast 2.0, gapped blastp set to default parameters. It will be appreciated that these sequence identity ranges are provided for guidance only; it is entirely possible that strongly significant homologs could be obtained that fall outside of the ranges provided.

Sperm ligand: For purposes of this discussion, “sperm ligand” means a protein expressed on the sperm membrane (either plasma or acrosomal) that interacts with a protein expressed on the oocyte plasma membrane and is involved in sperm-oocyte binding or fusion or oocyte activation. A sperm ligand may contain, but is not required to have, an integrin binding sequence.

Two-dimensional gel electrophoresis (2-DE): A method of separating mixtures of proteins with high resolution. In the first dimension, proteins are separated based on their isoelectric point. Proteins are then separated in a second dimension based on their molecular weight using standard SDS-PAGE. 2-DE may also be carried out with separation in the first dimension being based on protein molecular weight and separation in the second dimension based on their isoelectric point. Proteins separated using 2-DE can be detected using various methods, including by staining with a dye such as Coomassie blue, labeling with fluorescent dyes, or using an antibody labeled with a radioactive, fluorescent, or enzymatic tag. See, e.g. Ausubel et al. Short Protocols in Molecular Biology, 4^{th} Edition, Wiley, 1999, Chapter 10.

### Overview of Specific Embodiments

Provided herein are methods for increasing rates of oocyte fertilization using proteins from sperm that interact with the oocyte plasma membrane. Also provided are methods of preventing fertilization by using the identified sperm proteins to generate a contraceptive vaccine.

In specific embodiments, the method includes treating an oocyte with a purified sperm protein that interacts with the oocyte plasma membrane. The sperm protein may function in binding of sperm to the plasma membrane, promoting the fusion of the sperm and oocyte, or inducing oocyte activation to begin the embryo developmental program. In specific examples, the sperm protein contains an integrin binding sequence.

In a further embodiment, the method involves treating an oocyte with a purified sperm protein that induces oocyte activation. In a specific example, the oocyte can be fertilized in vitro by standard techniques. In further specific examples, the oocyte can be fertilized by intracytoplasmic sperm injection (ICSI), or the oocyte can be a recipient for nuclear transfer.

In another embodiment, the method involves treating an oocyte with a purified sperm protein that promotes sperm-oocyte fusion. In a particular example, the oocyte can be fertilized in vitro by standard techniques. In a further specific embodiment, the sperm protein can be a protein that has homology to the bacterial outer membrane protein family.

In other specific embodiments, the method involves inducing in a subject an immune response to at least one sperm protein, such that fertilization of oocytes is prevented. In particular examples, the sperm protein can be one that induces sperm binding to an oocyte, promotes sperm-oocyte fusion, or induces oocyte activation. In one embodiment, the method includes the administration of at least one purified polypeptide to a subject, such that an immune response sufficient to prevent oocyte fertilization is induced.

In one embodiment there is provided a method of increasing oocyte fertilization, which comprises treating an oocyte with a purified sperm protein, or fragment thereof, that interacts with the oocyte plasma membrane and promotes specific binding, sperm-oocyte fusion, or oocyte activation. In various examples, the purified sperm protein comprises an integrin-binding sequence. It is specifically contemplated that the oocyte in certain uses of the described methods is fertilized in vitro (for instance, by intracytoplasmic sperm injection) and/or the oocyte is a recipient for nuclear transfer.

Also provided are methods to prevent fertilization of an oocyte, which comprises inducing in a subject an immune response to at least one sperm protein that interacts with the oocyte plasma membrane and induces specific binding, sperm-oocyte fusion, or oocyte activation, such that fertilization of the oocyte is blocked. In example embodiments of these methods, induction of the immune response comprises administration of at least one purified polypeptide, comprising a sperm protein that interacts with the oocyte plasma membrane, in a pharmaceutically acceptable carrier, such that an immune response sufficient to prevent fertilization is generated.

Yet other described methods are methods to prevent fertilization of an oocyte, which methods involve treating an oocyte with a purified sperm protein, or fragment thereof, that interacts with the oocyte plasma membrane and inhibits or blocks specific binding, sperm-oocyte fusion, or oocyte activation, such that fertilization of the oocyte is blocked.

Details of specific aspects of methods to increase rates of oocyte fertilization and to prevent fertilization utilizing sperm proteins that interact with the oocyte plasma membrane are provided below. It will be recognized that the discussion herein is intended to provide representative examples and is not limiting.

We also have data indicating that G-proteins are functional in bovine oocyte development, as oocytes microinjected with the GDPβ[S] inhibitor do not cleave as often as control groups. Microinjection of 1 mM GDPβ[S], 2 mM GDPβ[S], and 4 mM GDPβ[S] followed by IVF resulted in 46.9% (76/162) cleavage, 26.7% (35/131) cleavage, and 11.7% (20/171) cleavage respectively. What effect GDPβ[S] might have on intracellular Ca²⁺ transients is yet to be determined. More specific inhibitors of G-protein subunits can be used to determine which subunits are specifically involved in fertilization pathways.

IV. Identification of Sperm Proteins that Interact with Oocyte Plasma Membrane

Sperm proteins that interact with the oocyte plasma membrane were identified herein using a method utilizing live sperm and oocytes. Sperm were labeled with a fluorescent dye, such as Cy2, Cy3, or Cy5. Labeled sperm were used to fertilize oocytes from which the zona pellucida were removed. Sperm-oocyte complexes were either immediately lysed or lysed following covalent crosslinking with a crosslinking agent, such as dibromobimane. Lysates were analyzed by 2-DE and protein spots from crosslinked and non-crosslinked lysates compared. Protein spots that shifted position upon crosslinking are presumed to have bound to a protein on the oocyte plasma membrane. These spots were picked and the proteins identified, for instance by mass spectrometry and comparison with protein databases.

Additional methods that can be used to identify protein-protein interactions are known in the art. These include but are not limited to, peptide display libraries (see, e.g. U.S. Pat. Nos. 5,223,409; 5,403,484; 5,571,698; and 5,837,500), two-hybrid systems (see, e.g., U.S. Pat. No. 5,283,173), co-immunoprecipitation, and affinity purification.

### V. Sperm Protein Expression and Purification

The expression and purification of proteins, such as a sperm ligand protein, can be performed using standard laboratory techniques. Examples of such methods are discussed or referenced herein. After expression, purified protein may be used for functional analyses, antibody production, diagnostics, and patient therapy, for instance.

Partial or full-length cDNA sequences, which encode for the subject protein, may be ligated into bacterial expression vectors. Methods for expressing large amounts of protein from a cloned gene introduced into Escherichia coli (E. coli) or baculovirus/Sf9 cells (or other expression system) may be utilized for the purification, localization and functional analysis of proteins. For example, fusion proteins consisting of amino terminal peptides encoded by a portion of a gene native to the cell in which the protein is expressed (e.g., an E. coli lacZ or trpE gene for bacterial expression) linked to a sperm ligand protein may be used to prepare polyclonal and monoclonal antibodies against these proteins. Thereafter, these antibodies may be used in various techniques and methods, for instance to purify proteins by immunoaffinity chromatography, in diagnostic assays, to quantitate the levels of protein and to localize proteins in tissues and individual cells by immunofluorescence, and so forth.

Intact native protein may also be produced in large amounts for functional studies and other applications. Methods and plasmid vectors for producing fusion proteins and intact native proteins in culture are well known in the art, and specific methods are described in Sambrook et al. (In Molecular Cloning: A Laboratory Manual, Ch. 17, CSHL, New York, 1989). Such fusion proteins may be made in large amounts, are easy to purify, and can be used to elicit antibody response. Native proteins can be produced in bacteria by placing a strong, regulated promoter and an efficient ribosome-binding site upstream of the cloned gene. If low levels of protein are produced, additional steps may be taken to increase protein production; if high levels of protein are produced, purification is relatively easy. Suitable methods are presented in Sambrook et al. (In Molecular Cloning: A Laboratory Manual, CSHL, New York, 1989) and are well known in the art. Often, proteins expressed at high levels are found in insoluble inclusion bodies. Methods for extracting proteins from these aggregates are described by Sambrook et al. (In Molecular Cloning: A Laboratory Manual, Ch. 17, CSHL, New York, 1989). Vector systems suitable for the expression of lacZ fusion genes include the pUR series of vectors (Ruther and Muller-Hill, EMBO J. 2:1791, 1983), pEX1-3 (Stanley and Luzio, EMBO J. 3:1429, 1984) and pMR100 (Gray et al., Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET-3 (Studiar and Moffatt, J. Mol. Biol. 189:113, 1986).

Fusion proteins may be isolated from protein gels, lyophilized, ground into a powder and used as an antigen. The DNA sequence can also be transferred from its existing context to other cloning vehicles, such as other plasmids, bacteriophages, cosmids, animal viruses and yeast artificial chromosomes (YACs) (Burke et al., Science 236:806-812, 1987). These vectors may then be introduced into a variety of hosts including somatic cells, and simple or complex organisms, such as bacteria, fungi (Timberlake and Marshall, Science 244:1313-1317, 1989), invertebrates, plants (Gasser and Fraley, Science 244:1293, 1989), and animals (Pursel et al., Science 244:1281-1288, 1989), which cell or organisms are rendered transgenic by the introduction of the heterologous cDNA.

For expression in mammalian cells, the cDNA sequence may be ligated to heterologous promoters, such as the simian virus (SV) 40 promoter in the pSV2 vector (Mulligan and Berg, Proc. Natl. Acad. Sci. USA 78:2072-2076, 1981), and introduced into cells, such as monkey COS-1 cells (Gluzman, Cell 23:175-182, 1981), to achieve transient or long-term expression. The stable integration of the chimeric gene construct may be maintained in mammalian cells by biochemical selection, such as neomycin (Southern and Berg, J. Mol. Appl. Genet. 1:327-341, 1982) and mycophenolic acid (Mulligan and Berg, Proc. Natl. Acad. Sci. USA 78:2072-2076, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

The cDNA sequence (or portions derived from it) or a mini gene (a cDNA with an intron and its own promoter) may be introduced into eukaryotic expression vectors by conventional techniques. These vectors are designed to permit the transcription of the cDNA in eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., Proc. Natl. Acad. Sci. USA 78:1078-2076, 1981; Gorman et al., Proc. Natl. Acad. Sci USA 78:6777-6781, 1982). The level of expression of the cDNA can be manipulated with this type of vector, either by using promoters that have different activities (for example, the baculovirus pAC373 can express cDNAs at high levels in S. frugiperda cells (Summers and Smith, In Genetically Altered Viruses and the Environment, Fields et al. (Eds.) 22:319-328, CSHL Press, Cold Spring Harbor, N.Y., 1985) or by using vectors that contain promoters amenable to modulation, for example, the glucocorticoid-responsive promoter from the mouse mammary tumor virus (Lee et al., Nature 294:228, 1982). The expression of the cDNA can be monitored in the recipient cells 24 to 72 hours after introduction (transient expression).

In addition, some vectors contain selectable markers such as the gpt (Mulligan and Berg, Proc. Natl. Acad. Sci. USA 78:2072-2076, 1981) or neo (Southern and Berg, J. Mol. Appl. Genet. 1:327-341, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., Mol. Cell Biol. 1:486, 1981) or Epstein-Barr (Sugden et al., Mol. Cell Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb, Virology 52:466, 1973) or strontium phosphate (Brash et al., Mol. Cell Biol. 7:2013, 1987), electroporation (Neumann et al., EMBO J 1:841, 1982), lipofection (Felgner et al., Proc. Natl. Acad. Sci USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70, 1987). Alternatively, the cDNA, or fragments thereof, can be introduced by infection with virus vectors. Systems are developed that use, for example, retroviruses (Bernstein et al., Gen. Engr'g 7:235, 1985), adenoviruses (Ahmad et al., J. Virol. 57:267, 1986), or Herpes virus (Spaete et al., Cell 30:295, 1982). Sperm ligand protein encoding sequences can also be delivered to target cells in vitro via non-infectious systems, for instance liposomes.

Using the above techniques, the expression vectors containing a sperm ligand gene sequence or cDNA, or fragments or variants or mutants thereof, can be introduced into human cells, mammalian cells from other species or non-mammalian cells as desired. The choice of cell is determined by the purpose of the treatment. For example, monkey COS cells (Gluzman, Cell 23:175-182, 1981) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

The host cell, which may be transfected with the vector of this disclosure, may be selected from the group consisting of E. coli, Pseudomonas, Bacillus subtilis, Bacillus stearothermophilus or other bacilli; other bacteria; yeast; fungi; insect; mouse or other animal; or plant hosts; or human tissue cells.

VI. Identification of Functional Activity of Sperm Proteins that Interact with Oocyte Plasma Membrane

Screening methods are provided, which can be used to identify and characterize the functional activity of sperm proteins that interact with the oocyte plasma membrane (or fragments or derivatives or analogs of such sperm ligand proteins). Three categories of sperm ligand proteins are described: those that participate in sperm binding to the oocyte plasma membrane; those that participate in promoting fusion of the sperm with the oocyte; and those that participate in the induction of oocyte activation following fertilization.

### Sperm-Oocyte Binding

Specific binding must occur between a sperm and an oocyte in order for fertilization to occur. Proteins (and other molecules) can be screened for their ability to bind to the oocyte plasma membrane by a competitive inhibition assay; conversely, the same or similar methods can be used to identify and characterize molecules that inhibit such binding. Sperm-oocyte binding can be monitored in vitro by visual inspection under light microscopy. Oocytes can be incubated with increasing amounts of purified sperm ligand proteins prior to fertilization in vitro. After a suitable period of incubation, such as 10, 20, 30, 40, 60 minutes or more, the binding status of the sperm and oocyte can be determined. Proteins that bind to the oocyte plasma membrane can be detected based on their ability to interfere with (or enhance) sperm-oocyte binding at increasing protein concentrations.

### Sperm-Oocyte Fusion

Following binding of a sperm to the oocyte, the plasma membranes of the two cells must fuse in order for the male genetic material to enter the oocyte and result in fertilization. Proteins can be screened for their ability to promote sperm-oocyte fusion by visualizing the presence of the sperm nucleus within the oocyte. Sperm ligand proteins that are identified as interacting with the oocyte plasma membrane, such as by a 2-DE assay, can be incubated with isolated sperm and oocytes, for example from cattle, sheep, goats, pigs, horses, mice, rats, non-human primates, rabbits, cats, or dogs, under IVF conditions. After a suitable period of incubation, such as 10, 20, 30, 40, 60 minutes or more, the fusion status of the sperm and oocyte can be determined and optionally quantified. Proteins (or other molecules) that increase the number of sperm-oocyte fusion events, or that decrease the amount of time for fusion to occur, may be considered promoters (or enhancers) of sperm-oocyte fusion. Proteins (or other molecules) that decrease the number of sperm-oocyte fusion events, or that increase the amount of time required for fusion to occur, may be consider inhibitors of sperm-oocyte fusions.

Sperm-oocyte fusion can be monitored by visualizing the presence of the sperm nucleus within the oocyte. Oocytes can be pre-loaded with a fluorescent DNA stain, including but not limited to, Hoechst 33258, Hoechst 33342, Hoechst 34580, and 4′,6-diamidino-2-phenylindole, dihydrochloride (DAPI). Oocytes can subsequently be incubated with live sperm, in the presence or absence of a purified sperm ligand protein (or peptide from such a protein, or an analog thereof), and monitored by fluorescent microscopy. The fluorescent labeling of the sperm nucleus by the DNA stain pre-loaded in the oocyte indicates that sperm-oocyte fusion has occurred.

### Oocyte Activation

Oocyte activation at fertilization is signaled by a series of intracellular calcium oscillations (Berridge and Galione, FASEB J. 2:3074-3082, 1988). Proteins and other molecules can be screened for their ability to induce (or inhibit) oocyte activation by monitoring the mobilization of intracellular calcium in an oocyte or by visualizing the formation of a pronucleus or by observation of cell division. Sperm proteins that are identified as interacting with the oocyte plasma membrane, such as by a 2-DE assay, can be incubated with isolated oocytes, for example oocytes from cattle, sheep, goats, pigs, horses, mice, rats, non-human primates, rabbits, cats, or dogs. After a suitable period of incubation, such as 10, 20, 30, 40, 60 minutes or more, the activation status of the oocyte can be determined. Proteins (or other molecules) that induce or enhance intracellular calcium mobilization or formation of a pronucleus or cell division may be considered to be promoters of oocyte activation. Proteins (or other molecules) that inhibit or prevent or reduce intracellular calcium mobilization or formation of a pronucleus or cell division may be considered inhibitors of oocyte activation.

The mobilization of intracellular calcium or the influx of calcium from outside the cell can be measured using standard techniques (e.g. Takahashi et al. Phys. Rev. 79:1090-1125, 1999). One method of intracellular Ca²⁺ detection is loading cells with a calcium sensitive fluorescent dye using standard methods, and measuring the change in Ca²⁺ levels using a fluorometer. Commonly used calcium indicators include analogs of BAPTA (1,2-bis(2-aminophenoxy)ethane-N,N,N′,N′-tetraacetic acid), such as Fura-2, Fluo-2, and Indo-1, which produce shifts in the fluorescent excitation or emission maxima upon binding calcium, and Fluo-3 and Calcium Green-2, which produce increases in fluorescence intensity upon binding calcium. Additional calcium indicator dyes include, but are not limited to, Quin-2, Fluo-4, Fluo-5N, Oregon green BAPTA, Calcium orange, Fura red, Rhod-2, Bis-fura-2, Mag-indo-1, Mag-fura-2, and BTC. See e.g., U.S. Pat. No. 5,516,911 and Takahashi et al.). It is known in the art that activation and emission wavelengths must be selected based on the calcium indicator chosen.

Activation can also be assessed by visualizing the formation of a pronucleus. Methods to detect the formation of a pronucleus are known in the art. For example, the pronucleus can be visualized by labeling the chromatin with a fluorescent dye, such as Hoechst 33342 and examining the cell by fluorescence microscopy. (See e.g. Miller et al. J. Cell Biol. 6:1289-1295, 2000). Cell division is easily detectable based on visual observation in conjunction with labeling the chromatin as described for pronucleus observation above.

### VII. Utilization of Sperm Ligand Proteins to Increase Fertilization Success

Disclosed herein are methods of using sperm ligand proteins which interact with the oocyte plasma membrane (and molecules derived such sperm ligand proteins) to increase rates of fertilization, for example in assisted reproductive technologies. Oocytes can be treated with at least one sperm ligand protein (or other molecule) that increases sperm binding to oocytes, sperm-oocyte fusion, or oocyte activation in order to achieve more efficient outcomes.

In particular examples, the sperm ligand proteins may contain at least one integrin binding sequence. Although there is diversity in the type of ligand that binds cells through integrins, there is a common element to the ligand motif. Most integrins bind to an element that contains an aspartic acid residue (RGD, ECD, LDV, KGD, RTD, and KQAGD). Many integrins recognize the RGD sequence, which appears in extracellular matrix (ECM) proteins and cell surface molecules, and has been implicated in fertilization. Examples of bovine sperm ligand proteins that contain an integrin binding domain include, but are not limited to angiotensin converting enzyme, heat shock protein 70, a protein with homology to bacterial outer membrane protein, inositol 1,4,5-triphsophate receptor type 3, and SMC3.

In one example, oocytes that are being fertilized by standard IVF can be incubated with one or more purified sperm protein ligand(s) described herein to increase rates of successful fertilization.

In certain embodiments, one or more purified sperm ligand proteins that promote oocyte activation are used to improve rates of fertilization in assisted reproductive techniques. In a particular example, oocytes that are fertilized by ICSI are incubated with purified sperm ligand protein(s) to increase fertilization success. In particular, fertilization by ICSI often fails due to a failure of oocyte activation. In one example, oocytes that have been injected with a sperm can be incubated with a sperm ligand that promotes oocyte activation.

In a further example, artificial oocyte activation is required to generate embryos by nuclear transfer. Rather than activation by current methods, such as treatment with calcium ionophore, ethanol, or electrical pulse, activation can be achieved by incubation of the oocyte following nuclear transfer with a sperm ligand that promotes activation. Activation that more closely mimics the process that occurs in vivo is expected to lead to more successful rates of development of nuclear transfer embryos.

In another example, purified sperm ligand proteins that promote sperm-oocyte fusion are used to improve rates of fertilization in IVF. In a particular example, oocytes that are fertilized by IVF are incubated with a sperm protein ligand that promotes sperm-oocyte fusion. In a particular example, the sperm ligand protein is a protein with homology to the bacterial outer membrane protein (BOMP) family. Of BOMP family proteins, invasin seems to be the best” match to the protein identified herein; another possibility is OmpA protein, which matches Azoarcus sp.. This protein is an outer membrane protein required for conjugation.

The identification of the bacterial outer membrane protein (BOMP) had the best RMS mass error score of 5.8504, it matched molecular weight exactly and estimated pI was the same.

### VIII. Methods to Prevent Fertilization Utilizing Sperm Ligand Proteins

Disclosed herein are methods of using purified sperm protein ligands and molecules derived therefrom to prevent fertilization, for instance by inducing an immune response that blocks sperm-oocyte interaction, fusion or oocyte activation. The general concept of immunocontraception is known in the art (see, e.g. U.S. Pat. Nos. 6,962,988; 7,056,515; and 7,094,547).

In a particular example, an immune response to at least one sperm ligand protein which interacts with oocyte plasma membrane is induced in a subject. In particular examples, the subject is a mammal, such as a human or a non-human animal (including but not limited to cattle, sheep, horses, pigs, rodents, goats, fowl, cats, and dogs).

The induction of the immune response can be generated by administration to a subject of an effective immunizing dose of at least one purified sperm ligand protein (or one or more epitopes from such a protein or proteins) in a pharmaceutically acceptable carrier. Routes of administration include but are not limited to, oral, intramuscular, intraperitoneal, intravenous, subcutaneous, intranasal, intravaginal, or any other standard route of immunization. An effective immunizing dose is one that is sufficient to produce an immune response to the antigen in a subject. It will be recognized by one of skill in the art that the effective immunizing dose will vary depending on factors such as the route of administration and the size and nature of the subject to be immunized, as well as the specific antigen and delivery system used. Antibody titer can be monitored following immunization to determine if a sufficient immune response has been generated.

The purified sperm protein ligands identified herein (or fragments thereof) can also be used to prevent (or reduce) fertilization by blocking (or inhibiting) specific binding, sperm-oocyte fusion, or oocyte activation, such that fertilization of the oocyte is blocked. Methods described herein can be used, or adapted, to characterize the sperm proteins (or fragments or derivates thereof) with regard to their ability to function to block oocyte fertilization.

### EXAMPLES

The following examples are provided to illustrate certain particular features and/or embodiments. These examples should not be construed to limit the invention to the particular features or embodiments described.

### Example 1

### Detection of Sperm Protein Ligands

The following example describes a method of detecting sperm protein ligands that interact with oocyte membrane proteins.

### Materials and Methods

### Sperm Binding to Oocytes

Frozen bovine semen was thawed, centrifuged on a 45% over 90% Percoll™ gradient, then washed once with Sperm TALP. Sperm was capacitated with heparin and acrosome reacted with lysophosphatidylcholine according to published procedures (Parrish et al., Gamete Res 24(4):403-413, 1989). We have determined that sperm treated in this manner are able to induce intracellular calcium transients typical of fertilization in zona-free bovine oocytes. Sperm were then labeled with a fluorescent dye (e.g., Cy2™, Cy3™ or Cy5™) and washed three times in Sperm TALP by centrifugation and removal of supernatant, to eliminate unbound dye. A final wash of sperm was performed in fertilization medium and the supernatant removed. A volume of 100 μl fertilization medium was added to the sperm pellet, resulting in a concentration of 625 million sperm per ml in suspension. Microdrops of sperm suspension were covered with warmed mineral oil and 300 zona-free oocytes were added to the each microdrop.

Bovine ovaries were collected from the local abattoir and oocytes from follicles between 3 and 8 mm were aspirated into 50 ml centrifuge tubes using an 18-gauge needle connected to a vacuum pump. Oocytes with intact layers of cumulus cells and evenly shaded cytoplasm were selected and washed in PB1 medium (described in Sessions et al., Mole. Reprod. Devel. 73:651-657, 2006) containing calcium and magnesium, supplemented with 3 mg/ml BSA (PB1+). Oocytes were then transferred into 500 μL of maturation medium; M199 medium containing 10% fetal bovine serum (FBS; HyClone Laboratories, Logan, Utah), 0.5 μg/ml FSH (Sioux Biochemicals, Sioux City, Iowa), 5 μg/ml LH (Sioux Biochemicals), 100 U/ml penicillin (HyClone Laboratories) and 100 μg/mL streptomycin (HyClone Laboratories) into four-well culture dishes (Nunc, Milwaukee, Wis.) and cultured at 39° C. in a humidified atmosphere of 5% CO₂ and air for 24 hours. At 24 hours after the initiation of maturation, oocytes were vortexed in 1 ml PB1+ to completely remove cumulus cells. Oocytes were moved up and down through a narrow-bore pipette in a 1% solution of pronase to remove zonae pellucidae (ZP). As soon as the ZP began to deform, oocytes were moved to a drop of PB1+ and moved up and down until the ZP were completely removed. Special care was taken not to overexpose the oocytes to the pronase solution. The denuded oocytes were washed extensively in PB1+ and placed in maturation medium for 6 hours in an incubator at 39° C., 5% CO₂, and humidified air. After recovery, oocytes were washed through 4 drops of PB1+ containing 3 mg/mL polyvinyl alcohol (PVA) rather than BSA.

Maximum sperm binding using this procedure occurs within 30 minutes. Sperm-oocyte complexes were then washed through PB1+ to remove unbound sperm. Complexes with sperm labeled by Cy3™ were placed in lysis buffer containing 8 M urea, 4% CHAPS, 40 mM Tris, and Complete™ Protease Inhibitor Cocktail (Roche Diagnostic, Manheim, Germany). Complexes with sperm labeled by Cy5™ were transferred to a solution containing 5 μM of the cross-linking reagent dibromobimane (bBBr) for 30 minutes after which complexes were washed extensively through 12 drops of PB1, then transferred to the lysis buffer. A control of unbound sperm labeled with Cy2™ and unbound oocytes was also lysed and run on the same gel. Unbound, unlinked, and linked lysates were all electrophoretically run on the same 2-D gel.

### 2-D Gel Electrophoresis

An 11 cm BioRad IPG strip was rehydrated with buffer containing 8M urea, 4% (w/v) CHAPS, 50 μg/mL DTT, 1% Pharmalyte™ and mixed labeled proteins at a combined volume of 185 μL. The strip was rehydrated overnight covered with mineral oil at room temperature. Isoelectric focusing was performed on a BioRad IPG Cell using electrode wicks each of which was hydrated with 8 μL of double de-ionized water. Focusing was performed for a total of 25,000 volt-hours. IPG strips were then equilibrated in buffer containing 100 mM Tris, 6 M urea, 30% glycerol, 2% (w/v) SDS, and 0.2 mg/mL DTT for 15 minutes. This equilibration was followed by a second equilibration in buffer containing 100 mM Tris, 6 M urea, 30% glycerol, 2% (w/v) SDS, and 0.022 mg/mL iodoacetamide for an additional 15 minutes. SDS PAGE was performed using a BioRad Criterion™ pre-cast gel on a 10-20% gradient using an SDS running buffer containing 25 mM Tris, 192 mM glycine, and 1% (w/v) SDS at a constant 200 volts. Gels were washed and stored in a fixing solution containing 40% methanol and 10% acetic acid.

### Fluorescence Imaging

The Typhoon Trio+™ fluorescence imager (GE Healthcare) was used to scan fluorescent labels at a pixel size of 100 μm. Protein spots on unbound, unlinked, and linked 2-D gels were compared to determine whether sperm and oocyte protein binding caused a shift on the gel, thus indicating some close interaction between sperm proteins and oocyte proteins.

### Results

We developed a novel technique to label live sperm or oocyte membrane proteins with a specific fluorescent dye. The gametes were allowed to interact, thus offering the best opportunity to identify membrane proteins functioning in their native state. It is very important to maintain the structural integrity of membrane proteins because removal of proteins from this environment could alter binding capacity. By labeling membrane proteins in live sperm and allowing them to fertilize in situ, we can see specific interactions and have higher confidence that these interactions are not an aberration resulting from the procedure. In some cases, following sperm binding to oocytes, the mixture was treated with a cross-linking agent prior to lysis and 2-DE. The cross-linking reagent serves the function of covalently linking proteins so that they remain together as a unit through lysis of the cells and 2-D gel analysis, which can be identified by mass spectrometry.

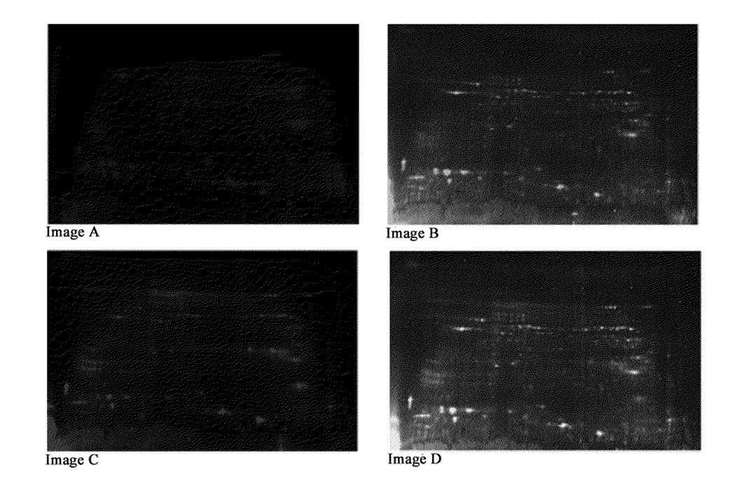

Binding and cross-linking of fluorescently labeled sperm to proteins on the plasma membrane of oocytes resulted in a shift in the position of labeled sperm proteins in a 2-D gel when compared with unbound sperm, and sperm proteins that were bound to oocytes, but not cross-linked to oocyte membrane proteins (FIG. 1). A 2-D gel containing sperm proteins that were not cross-linked to oocyte proteins (FIG. 1B) was superimposed onto a 2-D gel containing sperm proteins that were cross-linked to oocyte proteins (FIG. 1C). Any shift in position on the gel (FIG. 1D) is determined to be a result of the binding and cross-linking interaction between proteins.

### Example 2

### Identification of Sperm Proteins that Interact with Oocyte Plasma Membrane

The following example describes the identification of sperm protein ligands that interact with oocyte membrane proteins.

### Materials and Methods

### Identification of Sperm Proteins Using Mass Spectrometry

The protein spots that were identified based on the 2-D gel analysis of fluorescently labeled cell lysates (Example 1) were identified using three steps: in-gel digestion, mass spectrometry measurement, and database search.

In-Gel Digestion

The identified protein spots were excised and digested with trypsin. Protein gel spots were excised using an Ettan™ Spot Picker to select ˜1.5 mm pieces, and placed into 0.65-ml siliconized tubes. Gel pieces were washed three times with 100 μl of 25 mM ammonium bicarbonate/50% acetonitrile (pH 8.0), then dried in a vacuum centrifuge. Trypsin was added and the reaction was incubated 12 to 16 hours at 37° C. Peptides were extracted out of the gel using two volumes of 5% TFA/50% acetonitrile. Recovered peptides were concentrated by reducing the final volume of the extracts to ˜10 μl in a vacuum centrifuge.

### Mass Spectrometry

Peptide solutions from protein in-gel digestion were mixed 1:1 with matrix (10 mg/mL alpha-cyano-4-hydroxycinnamic acid in EtOH/AcN and spotted on a Micromass® target plate. After loading and firing the laser at the target, the peptide peaks were detected by matrix-assisted laser desorption/ionization (MALDI). The trypsin digestion peak was used as an internal calibration and adrenocorticotropic hormone fragment 18-39 (MH+2465.20) was used for the external calibration of the mass spectrometry peaks. Micromass® MassLynx™ 3.5 software was used for smoothing, subtracting, centering, and calibration of the spectra. For peptide mapping, a peptide MS peak list was generated by MassLynx™ 3.5.

### Database Search and Identification of Proteins

Peptide masses were compared with the sequences in the SwissProt cow database, the NCBI bovine database using Mascot (Perkins et al. Electrophoresis 20:3551-3567, 1999), and the cow database from the International Protein Index of the European Bioinformatics Institute FTP server. If the peptides matched with the theoretical peptides of a protein in the database with a significant score, the theoretical molecular weight and pI of the protein were compared with the experimental molecular weight and pI calculated from the 2-D gel. Protein identification was based on the peptide matches, searching match score, quality of the peptide map, intensity of match peak (18%-20% minimum), and similarity of experimental and theoretical molecular weight and pI.

### Results

Proteins that underwent a shift on 2-DE when cross-linked and non-cross-linked sperm were compared were selected for mass spectrometry analysis and identification. Proteins that were identified using mass spectrometry and database searching are shown in Table 1. Table 2 presents the same proteins as Table 1 and also includes the Genbank Accession Number of proteins that are homologous to the sperm proteins listed in Table 1. Six of the nineteen proteins identified contained at least one known integrin binding sequence. The methodology used in the described research involves labeling ONLY sperm protein with each of the CyDyes (different CyDye for each treatment), running all treatments within the same 2-D gel, taking individual images of each of the different treatments (which is enabled because a unique dye was used for each treatment, i.e., blue, red, green), overlaying the images and looking for spots in the “unbound” (no interaction with oocyte membrane proteins) treatment that moved in the “linked” treatment. This meant that the spot disappeared from its' location in this treatment because it was bound to something (i.e., the linker and oocyte membrane protein). We next went to the location where this spot in the “unbound” treatment was located and removed this protein and identified it with mass spec.

Several of these proteins are already known to be involved in fertilization (for instance angiotensin-converting enzyme, leucine aminopeptidase, heat shock protein 70, α-S1 casein, bacterial outer membrane proteins, and one hypothetical protein), which supports the validity of this identification system. Others had not previously been known to have a role in fertilization in any species; all are considered as proteins potentially involved in binding, fusion, or activation processes during fertilization.

TABLE 1
Sperm proteins identified as interacting with oocyte
membrane/receptors during fertilization, which were
identified by mass spectrometry.

| | | MW | |
|---|---|---|---|
| Putative Protein ID of Sperm | Integrin Binding | (kilodaltons | |
| Proteins | Sequence | or “kDa”) | pI |
| Angiotensin Converting Enzyme | LDV | 100-150 | 6.3 |
| Hsp 70 | LDV | 70 | 5.8 |
| Leucine Aminopeptidase | | 53 | 6.1 |
| Dihydrolipoyl dehydrogenase | | 54 | 7.6 |
| Enolase | | 47 | 6.8 |
| Malate dehydrogenase | | 30 | 9.7 |
| Bacterial Outer Membrane | ECD | 25 | 8.9 |
| Protein-like Protein | | | |
| Pdc109 | | 16 | 5.1 |
| 3-Hydroxybutyrate | | 32 | 5.7 |
| dehydrogenase | | | |
| Alpha S-1 Casein | | 25 | 5 |
| GTP-Binding Regulatory Go | | 40 | 5.5 |
| Alpha Chain | | | |
| FSH Receptor | | 78 | 6.8 |
| PREDICTED: hypothetical | RGD | 23.9 | 8.16 |
| protein [Rattus norvegicus] | | | |
| Glial Fibrillary Acidic Protein | | 47.9 | 5.26 |
| Potassium Voltage-Gated | | 96 | 9.01 |
| Channel | | | |
| Inositol 1,4,5-Trisphosphate | RGD/KGD | 305.7 | 6.08 |
| Receptor Type 3 | | | |
| T-cell Receptor Beta Chain | | 9.7 | 8.62 |
| Variable Segment | | | |
| Seminal Plasma Protein | | 15.9 | 4.91 |
| Precursor | | | |
| SMC3 | LDV/RTD | 142.3 | 8.54 |

TABLE 2
Sperm proteins identified in Table 1 and the Genbank
Accession Number of homologous proteins.

| | | | | Genbank |
|---|---|---|---|---|
| | | | | Accession No. |
| Putative Protein ID of Sperm | Integrin Binding | MW | | of homologous |
| Proteins | Sequence | (kDa) | pI | proteins |
| Angiotensin Converting Enzyme | LDV | 100-150 | 6.3 | 1919242A |
| Hsp 70 | LDV | 70 | 5.8 | 76650931 |
| Leucine Aminopeptidase | | 53 | 6.1 | 1127257 |
| Dihydrolipoyl dehydrogenase | | 54 | 7.6 | 76615133 |
| Enolase | | 47 | 6.8 | 87196501 |
| Malate dehydrogenase | | 30 | 9.7 | 1200100 |
| Bacterial Outer Membrane | ECD | 25 | 8.9 | 56311972 |
| Protein-like Protein | | | | |
| Pdc109 | | 16 | 5.1 | 494430 |
| 3-Hydroxybutyrate | | 32 | 5.7 | 44680136 |
| dehydrogenase | | | | |
| Alpha S-1 Casein | | 25 | 5 | 115646 |
| GTP-Binding Regulatory Go | | 40 | 5.5 | 71906 |
| Alpha Chain | | | | |
| FSH Receptor | | 78 | 6.8 | 544349 |
| PREDICTED: hypothetical | RGD | 23.9 | 8.16 | 34869072 |
| protein [Rattus norvegicus] | | | | |
| Glial Fibrillary Acidic Protein | | 47.9 | 5.26 | 27752368 |
| Potassium Voltage-Gated | | 96 | 9.01 | 12963342 |
| Channel | | | | |
| Inositol 1,4,5-Trisphosphate | RGD/KGD | 305.7 | 6.08 | 17432548 |
| Receptor Type 3 | | | | |
| T-cell Receptor Beta Chain | | 9.7 | 8.62 | 6687061 |
| Variable Segment | | | | |
| Seminal Plasma Protein | | 15.9 | 4.91 | 134452 |
| Precursor | | | | |
| SMC3 | LDV/RTD | 142.3 | 8.54 | 4235255 |

### Example 3

### Screening Sperm Ligands for Oocyte Activation Activity

This example describes methods to screen sperm ligands and molecules derived therefrom to identify those that can influence oocyte activation following sperm binding and fusion with an oocyte.

Sperm ligands that interact with oocytes are isolated and identified as described in Examples 1 and 2. Candidate proteins that may participate in oocyte activation are expressed in a heterologous expression system, such as E. coli and substantially purified by methods known in the art (see, e.g. Sambrook et al. In Molecular Cloning: A Laboratory Manual, Ch. 17, CSHL, New York, 1989).

Bovine oocytes are isolated and treated as described in Example 1. Oocytes are loaded with a fluorescent calcium probe, such as Fura-2. The oocytes are then incubated with a purified sperm ligand. Activation is monitored by detection of intracellular calcium oscillations. Once sperm proteins (or other molecules) that enhance oocyte activation are identified, the time of incubation and molecule concentration can be optimized by testing a variety of treatment conditions.

### Example 4

### Improved In vitro Fertilization by Assisted Oocyte Activation

This example describes use of sperm ligands to achieve improved rates of in vitro fertilization by assisted oocyte activation.

### Intracytoplasmic Sperm Infection

Intracytoplasmic sperm injection (ICSI) is a technique that generates an embryo in vitro by direct injection of a sperm into an oocyte. However, high rates of fertilization failure occur even when a sperm is successfully injected, due to a failure of oocyte activation (Yamano et al. J. Med. Invest. 47:1-8, 2000). Successful development of embryos generated by ICSI has been achieved by oocyte activation using artificial stimuli such as calcium ionophores or protein synthesis inhibitors (Yamano et al.). However, the potential cytotoxic, teratogenic, and mutagenic properties of these agents has limited their use in ICSI. Sperm proteins that naturally promote oocyte activation offer a more biological means of inducing oocyte activation following ICSI.

Oocytes and sperm are obtained and ICSI is carried out according to standard methods (Hewitson et al. Biol. Reprod. 55:271-280, 1996; Palermo et al. Lancet 340:17-18, 1992; Sutovsky et al. Hum. Reprod. 14:2301-2312, 1996; Van Steirteghern et al. Hum. Reprod. 8:1061-1066, 1993). Following or concurrent with injection of the sperm into the oocyte, a purified sperm protein (or molecule derived therefrom) shown to induce oocyte activation is added to the incubation medium. This results in increased rates of the fertilization.

### Nuclear Transfer

Nuclear transfer (NT) has been successfully utilized to produce cloned offspring in a number of mammalian species, including sheep, cattle, pigs, goats, and mice. Despite these successes, the process is very inefficient, with only a portion of the clones developing to the blastocyst stage in vitro, and only a portion of those blastocysts surviving to term following implantation in a host animal. One variable that may contribute to the low success rate of NT is the method of activating the oocyte following the transfer of donor genetic material. Current methods of oocyte activation in NT include treatment with a calcium ionophore, ethanol, direct current pulses, or injection of fertilized oocyte cytoplasm. With the provision in this disclosure of sperm protein ligands (and molecules derived therefrom) that enhance or induce oocyte activation, more biological methods of oocyte activation in NT are now enabled.

Methods of NT are well known in the art (see e.g. Stice et al., Theriogenology 49:129-138, 1998; Solter, Nature 394:315-316, 1998; Wakayama et al., Nature 394, 369-374, 1998; Wells et al., Biol. Reprod. 57:385-393, 1997; Wilmut et al., Nature 385:810-813, 1997). NT is performed according to a standard method, with the exception that activation of the oocyte is achieved by incubation of the oocyte (either before or after transfer of donor genetic material) with a sperm ligand (or molecule derived therefrom) that has been shown to cause oocyte activation, for instance using the methods described in Example 3.

### Example 5

### Screening Sperm Ligands for Sperm-Oocyte Fusion Activity

This example describes methods for screening sperm ligands (and molecules derived therefrom) to identify and/or characterize those that promote sperm-oocyte fusion.

Sperm ligands that interact with oocytes are isolated and identified as described in Examples 1 and 2. Candidate proteins that may participate in sperm-oocyte fusion are expressed in a heterologous expression system, such as E. coli and substantially purified by methods known in the art (see, e.g. Sambrook et al. In Molecular Cloning: A Laboratory Manual, Ch. 17, CSHL, New York, 1989).

Bovine oocytes are isolated, for instance as described in Example 1. Oocytes are loaded with a fluorescent DNA stain, such as DAPI or Hoechst 33258, for instance by inclusion of the stain in the oocyte incubation medium for 15 minutes. Sperm and oocytes are co-incubated for an appropriate period of time (e.g., 30 minutes) either in the presence or absence of a purified sperm ligand. Sperm-oocyte fusion is scored by detecting sperm nuclei fluorescently labeled by DNA stain transfer from the pre-loaded oocyte. See, e.g., Miller et al. J. Cell Biol. 149:1289-1295, 2000.

At low concentrations, sperm ligands that promote sperm-oocyte fusion are expected to enhance sperm fusion with the oocyte. At high concentrations, these ligands are expected to decrease the rate of sperm-oocyte fusion by blocking sperm-oocyte interaction through competitive inhibition. Following identification of sperm ligands involved in sperm-oocyte fusion, titration experiments can be carried out to determine the optimal protein concentration to promote (or inhibit) fusion.

### Example 6

### Sperm Ligands that Promote Sperm-Oocyte Fusion

The following example describes the use of sperm ligands (or molecules derived therefrom) that promote sperm-oocyte fusion to enhance oocyte fertilization.

In situations where standard IVF is not successful, (e.g. due to failure of sperm to bind to or fuse with an oocyte), ICSI is considered. ICSI may be avoided in some cases if a defect in sperm-oocyte fusion can be overcome.

Oocytes and sperm are isolated and IVF is carried out according to standard methods known in the art. A purified sperm ligand (or molecule derived therefrom) which has been shown to promote sperm-oocyte fusion (for instance, using the method in Example 6) is included in the medium during the incubation of sperm and oocytes. This is expected to improve the rate of successful fertilization.

The mass spectrum from a particular protein identified in the 2-DE screen (Examples 1 and 2) resembles proteins from the bacterial outer membrane (BOMP) protein family, and is proposed to be a similar protein that is in the bovine model but has not been previously characterized.

The protein identified in our studies contains an integrin binding sequence (ECD; Table 1). The oocyte is a non-phagocytic cell that must fuse or uptake the sperm cell after binding. BOMP proteins are involved in the process of invasion of enteropathogenic bacteria into non-phagocytic cells (Alrutz and Isberg, Proc Natl Acad Sci USA 95(23):13658-13663 1998). Invasin, a BOMP protein, mediates the uptake of bacteria and requires high affinity binding to β1 integrin receptors on the host eukaryotic cell. Alrutz and Isberg (1998) demonstrated that invasin-mediated uptake of bacterium into eukaryotic cells also required FAK. In this study, a dominant interfering form of FAK significantly reduced the amount of bacterial uptake. Additionally, cultured cells expressing interfering SRC kinase variants exhibited reduction in bacterial uptake. We have demonstrated the involvement of both FAK and Src kinases in fertilization as well as the presence of β1 integrins on bovine oocytes Pate et al., Mol. Reprod. Dev., E-pub Oct. 12, 2006c) and the role of integrins in bovine sperm-oocyte interactions leading to oocyte activation and development (Campbell et al., Biol Reprod 62(6):1702-1709, 2000; Sessions et al., Mol Reprod Dev 73(5):651-657, 2006; White et al., Mol. Reprod. Devel. 74(1):88-96, 2006). Invasin is a bacterial protein that mediates the uptake of bacterial cells into non-phagocytic eukaryotic cells. Although this specific protein (invasin or other BOMP proteins) has not been identified in the bovine model, it seems plausible that a bovine sperm ligand that is similar in form and function is present and mediates some sperm-oocyte interactions.

### Example 7

### Use of Sperm Ligands for Contraceptive Vaccines

This example describes the use of purified sperm ligands (or molecules derived therefrom, including for instance isolated epitopes) as immunogens for contraceptive vaccines.

Sperm ligands that interact with oocytes are isolated and identified as described in Examples 1 and 2. Proteins that are candidates for contraceptive vaccines are expressed in a heterologous expression system, such as E. coli and substantially purified by methods known in the art (see, e.g. Sambrook et al. In Molecular Cloning: A Laboratory Manual, Ch. 17, CSHL, New York, 1989).

Antibodies to epitopes from sperm ligands may be produced using standard procedures described in a number of texts, including Harlow and Lane (Antibodies, A Laboratory Manual, CSHL, New York, 1988). The determination that a particular agent binds substantially only to the specified protein may readily be made by using or adapting routine procedures. One suitable in vitro assay makes use of the Western blotting procedure (described in many standard texts, including Harlow and Lane (Antibodies, A Laboratory Manual, CSHL, New York, 1988). Western blotting may be used to determine that a given protein antibody, binds substantially only to the protein that was used as the immunogen.

Antibodies that block or inhibit fertilization can be detected using an IVF system. Bovine oocytes and sperm can be isolated as described in Example 1. Oocytes are pre-incubated with an antibody against a sperm ligand prior to the addition of sperm. Antibodies that prevent or reduce fertilization are candidates for a contraceptive vaccine.

A subject can be immunized with one or more sperm ligand polypeptides in order to block conception. An effective immunizing dose is administered to the subject, such that the subject produces an immune response to the antigen which is sufficient to block contraception. The generation of an immune response is determined by standard methods to determine antibody titer, such as ELISA.

In view of the many possible embodiments to which the principles of the disclosure and examples may be applied, it will be recognized that the illustrated embodiments are only examples of the invention and are not to be taken as limiting its scope.

We claim:

1. A method to prevent fertilization of an oocyte, comprising:
treating the oocyte with a purified sperm protein, or fragment thereof, that interacts with the oocyte plasma membrane and inhibits or blocks specific binding, sperm-oocyte fusion, or oocyte activation, such that fertilization of the oocyte is blocked.

2. The method of claim 1, wherein said purified sperm protein further comprises an integrin binding sequence.

3. The method of claim 1, wherein said purified sperm protein further comprises an integrin binding sequence, wherein the integrin binding sequence further comprises an ECD motif.

4. The method of claim 1, wherein said purified sperm protein further comprises an integrin binding sequence, wherein the integrin binding sequence further comprises an ECD motif, and wherein said purified sperm protein has a molecular weight of about 25 kDa and a pI of about 8.9.

5. The method of claim 1, wherein said purified sperm protein further comprises a protein having at least 95% identity to the bacterial outer membrane protein-like protein of Table 1.

6. The method of claim 5, wherein said purified sperm protein further comprises an integrin binding sequence.

7. The method of claim 5, wherein said purified sperm protein further comprises an integrin binding sequence, wherein the integrin binding sequence further comprises an ECD motif.

8. The method of claim 5, wherein said purified sperm protein further comprises an integrin binding sequence, wherein the integrin binding sequence further comprises an ECD motif, and wherein said purified sperm protein has a molecular weight of about 25 kDa and a pI of about 8.9.

9. The method of claim 1, wherein the purified sperm protein further comprises the bacterial outer membrane protein-like protein of Table 1.

10. The method of claim 9, wherein said bacterial outer membrane protein-like protein further comprises an integrin binding sequence.

11. The method of claim 9, wherein said bacterial outer membrane protein-like protein further comprises an integrin binding sequence, wherein the integrin binding sequence further comprises an ECD motif.

12. The method of claim 9, wherein said bacterial outer membrane protein-like protein further comprises an integrin binding sequence, wherein the integrin binding sequence further comprises an ECD motif, and wherein said purified sperm protein has a molecular weight of about 25 kDa and a pI of about 8.9.

13. The method of claim 1, wherein said oocyte further comprises a bovine oocyte.

14. The method of claim 2, wherein said oocyte further comprises a bovine oocyte.

15. The method of claim 4, wherein said oocyte further comprises a bovine oocyte.

16. The method of claim 5, wherein said oocyte further comprises a bovine oocyte.

17. The method of claim 8, wherein said oocyte further comprises a bovine oocyte.

18. The method of claim 9, wherein said oocyte further comprises a bovine oocyte.

19. The method of claim 12, wherein said oocyte further comprises a bovine oocyte.
